(19) United States
(12) Patent Application Publication
(10) Pub. No.: US 2025/0281032 A1
(43) Pub. Date: Sep. 11, 2025

(54) STEERABLE ORTHOPEDIC ENDOSCOPIC SYSTEM WITH NAVIGATION FUNCTION

(71) Applicant: THE FIRST AFFILIATED HOSPITAL OF CHONGQING MEDICAL UNIVERSITY, Chongqing (CN)

(72) Inventors: Wei HUANG, Chongqing (CN); Chen ZHAO, Chongqing (CN); Guifeng XIA, Chongqing (CN); Ning HU, Chongqing (CN); Wei SHUI, Chongqing (CN)

(21) Appl. No.: 19/068,057

(22) Filed: Mar. 3, 2025

(30) Foreign Application Priority Data
Mar. 5, 2024 (CN) 202410247234.X
Sep. 9, 2024 (CN) 202411256528.5

(51) Int. Cl. A61B 1/005; A61B 1/00; A61B 1/317

(52) U.S. Cl. CPC A61B 1/009; A61B 1/00057; A61B 1/00195; A61B 1/0057; A61B 1/317

(57) ABSTRACT

The present disclosure belongs to the technical field of orthopedic surgery, and in particular, to a steerable orthopedic endoscope system with navigation function. The system includes a handle, an endoscope body, a rotating adjustment component, an angle detection component, a processor, an optical navigation component, and an electromagnetic navigation component. The endoscope body includes a rod portion, a bendable portion, and a lens. The bendable portion is internally provided with bending sections. The rotating adjustment component includes a rotating wheel and two pull wires. The angle detection component includes an marker arranged on the rotating wheel and a detection device configured to identify the marker. The processor is electrically connected to the angle detection component and the rotating adjustment component.

### CROSS-REFERENCE TO RELATED APPLICATIONS

This application claims priority to Chinese Patent Application 202410247234.X, filed on Mar. 5, 2024, and entitled “ENDOSCOPE WITH VARIABLE ANGLE”, and Chinese Patent Application 202411256528.5, filed Sep. 9, 2024, and entitled “STEERABLE ORTHOPEDIC ENDOSCOPE SYSTEM WITH NAVIGATION FUNCTION”. Chinese Patent Application 202410247234.X and Chinese Patent Application 202411256528.5 are incorporated herein by reference in their entireties.

### FIELD

The present disclosure relates to the technical field of orthopedic surgery, and in particular, to a steerable orthopedic endoscope system with navigation function.

### BACKGROUND

With the deepening of minimally invasive concepts, the use of endoscopy in orthopedic surgery has become an important trend in development of surgical technologies. Orthopedic endoscopes allow orthopedic surgeon to perform intraoperative diagnosis and procedures through minimally invasive incisions, which greatly improves patient satisfaction and reduces medical costs. However, a current orthopedic endoscope (arthroscopes and foraminoscopes) typically features a rigid cylindrical design with a fixed field of view angle, which makes it difficult to adapt to irregular surgical environment. This limitation results in blind spots and restricted operational areas during surgery. In addition, current endoscopic systems are lack of navigation designs, forcing surgeons to rely solely on their interpretation of endoscopic anatomical structures viewed through the endoscope to estimate the spatial positioning of the lens and instruments. This technology has a steep learning curve and carries a high risk of iatrogenic injury. Therefore, there is an urgent clinical need for an orthopedic endoscope with variable observation angles and navigation functionality to support various surgeries, including joint surgery, spine surgery, and trauma surgery.

### SUMMARY

To eliminate surgical blind spots and restricted areas in orthopedic endoscope surgery, shorten a learning period of the orthopedic endoscope surgery, and improve surgical safety and quality, the present disclosure provides a steerable orthopedic endoscope system with navigation function. The endoscopic system can obtain pose information of a lens based on a rotation angle of a rotating wheel, and co-locate the lens through optical magnetic composite, which facilitates observing and operating by using the orthopedic endoscope system more accurately, reduces medical safety risks, shortens a learning curve of a doctor, and improves surgical quality.

Embodiments of the present disclosure provide a steerable orthopedic endoscope system with navigation function, including:
a handle;
an endoscope body, comprising a rod portion connected to the handle, a bendable portion located at a front end of the rod portion, and a lens located at a front end of the bendable portion, wherein the bendable portion is internally provided with bending sections, whereby the bendable portion is capable of bending at different angles;
a rotating adjustment component, comprising a rotating wheel and two pull wires arranged on two sides in a radial direction of the rotating wheel respectively, wherein the two pull wires are connected to the bending sections to adjust bending degree of the bendable portion;
an angle detection component, comprising a marker arranged on the rotating wheel and a detection device configured to identify the marker, wherein the detection device obtains a rotation angle of the rotating wheel by detecting changes of the marker; and
a processor, electrically connected to the angle detection component and the rotating adjustment component, wherein the processor receives rotation angle information obtained by the angle detection component, and calculates a pose of the lens relative to the rod portion according to the rotation angle information, and the pose comprises coordinates and a direction angle of the lens.

In some embodiments, an operation in which the processor calculates the pose of the lens relative to the rod portion according to the rotation angle information comprises:

Determining an x-axis coordinate (x) and a y-axis coordinate (y) of the lens according to the following mathematical expression:

$$y = {\sum }_{j = 0}^{n} {a}_{j} \cdot {x}^{j} = f ( x )$$

$$x = {\sum }_{j = 0}^{n} {b}_{j} \cdot {θ}^{j} = g ( θ )$$

wherein n is a number of terms in a polynomial, aⱼ and bⱼ are respectively coefficients of a j^{th} item in a vertical coordinate and a horizontal coordinate, and 0 is the rotation angle of the rotating wheel.

In some embodiments, the operation in which the processor calculates the pose of the lens relative to the rod portion according to the rotation angle information further comprises:
determining a normal at the lens according to the following mathematical expression, and taking an angle of the normal as the direction angle of the lens:

$$y = \frac{{x}_{i} - x}{{k}_{i}} + {y}_{i}$$

wherein xᵢ=g(θᵢ), kᵢ=f′(g(θᵢ)), and yᵢ=f(g(θᵢ)).

In some embodiments, an optical navigation component, wherein the processor adjusts an origin of the optical navigation component based on pose information of the lens, whereby a field of view of the optical navigation component is with the same as a field of view of the lens.

In some embodiments, the optical navigation component comprises a reference frame and a reflection sphere arranged on the handle, and the electromagnetic navigation component comprises an electromagnetic positioning sensor arranged at the front end of the bendable portion.

In some embodiments, the optical navigation component includes a plurality of the reflection spheres, and a number of the reflection spheres is three or more.

In some embodiments, the marker is a grating, and the detection device is a sensor that matches the grating.

In some embodiments, the rotating wheel is driven by a motor, and the motor is embedded into the handle.

In some embodiments, a button configured to start or stop the motor is arranged on the handle.

In some embodiments, the steerable orthopedic endoscope system with navigation function includes an electromagnetic navigation component, providing reference coordinates for the lens.

In some embodiments, n, aⱼ and bⱼ are determined by fitting a plurality of calibration results.

By using the steerable orthopedic endoscope system with navigation function according to the present disclosure, the orthopedic endoscope system controls the pull wires to adjust a bending angle of the bendable portion through the rotating wheel, the angle detection component detects and sends the rotation angle of the rotating wheel to the processor, the processor calculates the pose information of the lens relative to the rod portion through information of the rotation angle, and the orthopedic endoscope system can co-locate the lens based on the pose information in cooperation with optical magnetic composite, which facilitates observing and operating by using the orthopedic endoscope system more accurately, reduces medical safety risks, shortens a learning curve of a doctor, and improves surgical quality.

### BRIEF DESCRIPTION OF THE DRAWINGS

FIG. 1 is a schematic structural diagram of a steerable orthopedic endoscope system with navigation function.

FIG. 2 is a schematic structural diagram of the bendable portion in some embodiments.

FIG. 3 is a partial explosion diagram of the bendable portion in some embodiments.

FIG. 4 is a schematic structural diagram of the bendable section in some embodiments.

FIG. 5 is a block diagram showing various functional elements of an exemplary steerable orthopedic endoscope system.

In the drawing: 10, lens; 20, rod portion; 21, bendable portion; 211, bending section; 212, through-hole; 213, interior space; 214, pin; 30, handle; 40, rotating wheel; 41, pull wire; 50, grating; 51, angle detection component; 52, sensor; 60, motor; 61, button; 70, reference frame; 71, reflection sphere; 72, optical navigation component; 73, IR Camera; 81, electromagnetic navigation component; 80, electromagnetic positioning sensor and 90, processor.

### DETAILED DESCRIPTION

To make purposes, technical solutions, and advantages of the present disclosure clearer, the present disclosure is described below in detail in some embodiments with reference to an accompanying drawing and embodiments. It is to be understood that specific embodiments described here are merely used to explain the present disclosure and are not intended to limit the present disclosure.

Structures, proportions, sizes, and the like drawn in the drawing of the present specification are merely used to cooperate with the contents disclosed in the specification for those familiar with the technology to understand and read, and are not intend to limit the limitation conditions under which the present disclosure can be implemented. Any modifications of the structures, changes in proportion relationships or adjustment of the sizes still fall within the scope that can be covered by the technical content disclosed by the present disclosure without affecting the efficacy and the purpose that can be achieved by the present disclosure.

Orientations or positional relationships indicated by terms “upper”, “lower”, “left”, “right”, “middle”, “longitudinal”, “transverse”, “horizontal”, “inner”, “outer”, “radial”, “circumferential” and the like are the orientations or positional relationships shown based on the accompanying drawing, and are merely for the convenience of describing, rather than indicating or implying that devices or elements referred to need to have particular orientations, and constructed and operated in particular orientations. Thus, it cannot be construed as a limitation to the present disclosure. In addition, terms “first” and “second” are merely used for description, and cannot be understood as indicating or implying relative importance.

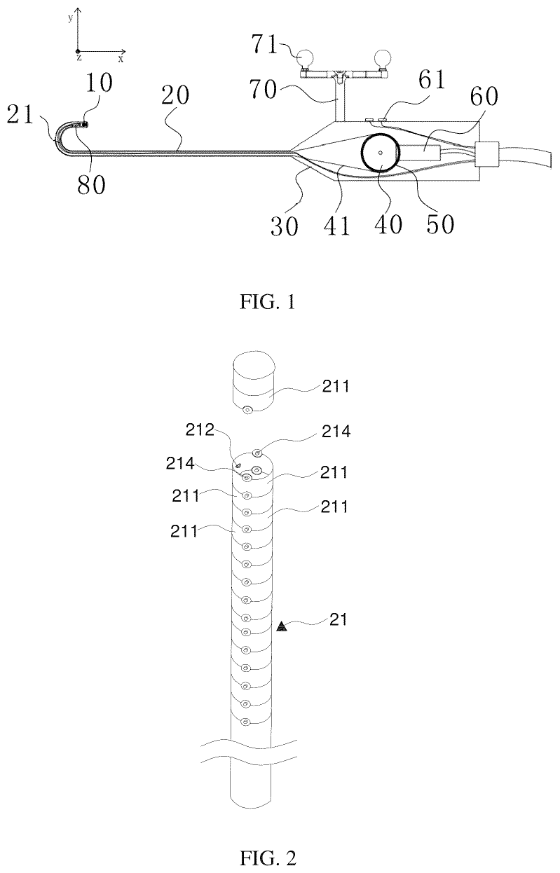

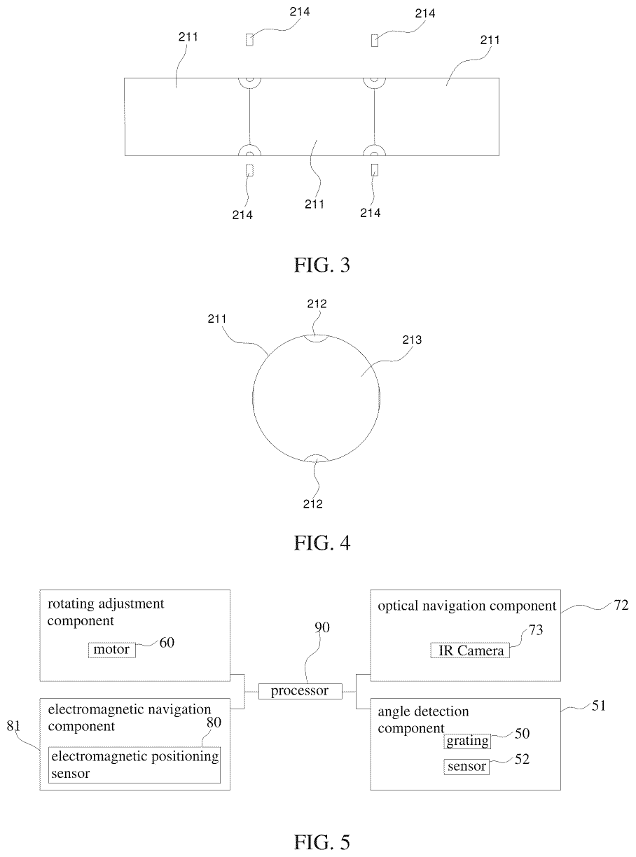

As shown in FIG. 1, the present disclosure discloses a steerable orthopedic endoscope system with navigation function, including a handle 30, an endoscope body, a rotating adjustment component, an angle detection component, and a processor 90. The endoscope body includes a rod portion 20 connected to the handle 30, a bendable portion 21 located at a front end of the rod portion 20, and a lens 10 located at a front end of the bendable portion 21. The bendable portion 21 is internally provided with bending sections 211, whereby the bendable portion 21 is capable of bending at different angles, which ensures that the lens 10 can meet visual field requirements at different positions within a human body. In some embodiments, bending sections 211 are linked together to form one line in the axial direction of the bendable portion 21. Each of the bending sections 211 includes through-holes 212 and an interior space 213. The through-holes 212 are provided in a manner to extend in the axial direction of the bendable portion 21, and allow pull wires 41 to be inserted therethrough. The interior space 213 is provided in a manner to extend in the axial direction of the bendable portion 21. Each pair of adjacent bending section 211 is coupled together by a pair of pins 214, thereby each bending sections 211 is connected to its adjacent bending sections 211, such that each bending section 211 is swingable about the axis of the pair of pins 214 (i.e., swingable about a swing axis). The swing axes of the bending sections 211 are configured to be parallel to each other. That is, the bendable portion 21 is configured to be bendable in respective directions of the swing axes of the bending sections 211.

The rotating adjustment component in this embodiment includes a rotating wheel 40 and two pull wires 41 arranged on two sides in a radial direction of the rotating wheel 40 respectively. The two pull wires 41 are connected to the bending sections 211. The rotating wheel 40 drives the bending sections 211 to move through the pull wires 41, so as to adjust bending degree of the bendable portion 21. During rotating of the rotating wheel 40, the pull wire 41 on one side of the rotating wheel 40 is tightened, and the pull wire 41 on the other side of the rotating wheel 40 is loosened, whereby the bendable portion 21 bends towards the side where the pull wire 41 is tightened. In some embodiments, the rotating adjustment component further includes a motor 60. The rotating wheel 40 is driven by the motor 60. In some embodiments, the motor 60 is a servo motor. The motor 60 is controlled to start or stop by a button 61 on the handle 30. The motor 60 is embedded into the handle 30. It is to be noted that, a method for controlling the bendable portion 21 to bend by using the rotating wheel 40, the pull wires 41, and the bending sections 211 is a conventional technology. Therefore, specific structures and principles are not described in detail in this embodiment.

The angle detection component 51 in this embodiment includes an marker arranged on the rotating wheel 40 and a detection device configured to identify the marker. The detection device obtains a rotation angle of rotating wheel 40 by detecting changes of the marker. In some embodiments, the marker is a grating 50. The detection device is a sensor 52 that cooperates with the grating 50. The grating 50 in cooperation with the sensor 52 has high measurement accuracy and high stability. In some embodiments, the marker is an optical structure typically includes periodic patterns, i.e., a grating with Moire fringes.

In some embodiments, the marker may be a conductor arranged on the rotating wheel 40, and a corresponding detection device is an ejector pin. The ejector pin is in contact with the conductor to form an electrical circuit to obtain the rotation angle of the rotating wheel 40. It is to be noted that a method for detecting the rotation angle of an object by using the grating 50 and the sensor is a conventional technology, which is not described in detail in this disclosure.

In some embodiments, the processor 90 is electrically connected to the angle detection component 51 and rotating adjustment component. Furthermore, the processor 90 is electrically connected to the motor 60.

The processor 90 receives rotation angle information obtained by the angle detection component, and calculates a pose (coordinates and a direction angle) of the lens 10 relative to the rod portion 20 according to the rotation angle information. It is to be noted that the lens 10 is controlled through the rotating wheel 40, the pull wires 41, and the bending sections 211. The lens 10 is constrained to move within a plane, that is, the lens 10 can only move in a plane where a certain radial direction of the rod portion 20 is located, so a numerical value of a z-axis coordinate of the lens 10 relative to the rod portion 20 is 0, and coordinates of the lens 10 relative to the rod portion 20 are (x, y, 0). This embodiment is mainly to calculate an x-axis coordinate and a y-axis coordinate of the lens 10 relative to the rod portion 20. The processor 90 in this embodiment may be a conventional STM32F4 or STM32F7 series single chip microcomputer, and the like.

A method that the processor calculates a pose of the lens 10 relative to the rod portion 20 according to the rotation angle information is as follows:

Firstly, a polynomial function curve of a trajectory of the lens 10 is set. A mathematical expression of the x-axis coordinate and the y-axis coordinate of the lens 10 relative to the rod portion 20 is represented by formula (1) as follows:

$$y = {\sum }_{j = 0}^{n} {a}_{j} \cdot {x}^{j} = f ( x ) \qquad ( 1 )$$

where n is a number of terms in a polynomial, and aⱼ is a coefficient of a j^{th} item in a vertical coordinate and is determined by fitting a plurality of calibration results. In some embodiments, the vertical coordinate is the y-axis coordinate and the horizontal coordinate is the x-axis coordinate. Therefore, in some embodiments, x is abscissa and y is ordinate.

Secondly, a polynomial function of the x-axis coordinate of the lens 10 and a rotation angle θ of the rotating wheel 40 is set. When θ>0, as the rotation angle θ increases, coordinates of the lens 10 are monotonically invariant in an x-axis direction when the rotation angle θ>0, x=g(θ) is set, the x-axis coordinate of the lens 10 may form a polynomial function with respect to the rotation angle θ, which is represented by formula (2) as follows:

$$x = {\sum }_{j = 0}^{n} {b}_{j} \cdot {θ}^{j} = g ( θ ) \qquad ( 2 )$$

where bⱼ is a coefficient of a j^{th} item in the horizontal coordinate. θ^{j} is a coefficient of the j^{th} item in the rotation angle, which is determined by fitting a plurality of calibration results.

In some embodiments, specific calculation methods for aⱼ and θ^{j} are as follows:

First, a mapping relationship between coordinates (xᵢ, yᵢ, 0) of the lens 10 relative to the rod portion 20 and the rotation angle θ of the rotating wheel 40, a single rotation angle of the rotating wheel 40 is set as Δα, when the rotating wheel is rotated for an i^{th} time, the rotation angle of the rotating wheel 40 is θᵢ=i·Δα, and the coordinates (xᵢ, yᵢ, 0) of the lens 10 relative to the rod portion 20 at this moment are recorded, where i=0, 1, 2, . . . , m.

After data is counted for m+1 times, list data of the rotation angle θ of the rotating wheel 40 and the coordinates (xᵢ, yᵢ, 0) of the lens 10 is obtained, which is represented by formula (3) as follows:

$$\{ \begin{matrix}{y}_{0} - {a}_{0} + {a}_{1} \cdot {x}_{0} + {a}_{2} \cdot {x}_{0}^{2} + … + {a}_{n} \cdot {x}_{0}^{n} \\ {y}_{1} - {a}_{0} + {a}_{1} \cdot {x}_{1} + {a}_{2} \cdot {x}_{1}^{2} + … + {a}_{n} \cdot {x}_{1}^{n} \\ {y}_{2} - {a}_{0} + {a}_{1} \cdot {x}_{2} + {a}_{2} \cdot {x}_{2}^{2} + … + {a}_{n} \cdot {x}_{2}^{n} \\ ⋮ \\ {y}_{m} - {a}_{0} + {a}_{1} \cdot {x}_{m} + {a}_{2} \cdot {x}_{m}^{2} + … + {a}_{n} \cdot {x}_{m}^{n}\end{matrix} \qquad ( 3 )$$

Then, formula (3) is converted into a matrix form, which is represented by formula as follows:

$$[ \begin{matrix}{y}_{0} \\ {y}_{1} \\ {y}_{2} \\ ⋮ \\ {y}_{m}\end{matrix} ] = [ \begin{matrix}1 & {x}_{0} & {x}_{0}^{2} & … & {x}_{0}^{n} \\ 1 & {x}_{1} & {x}_{1}^{2} & … & {x}_{1}^{n} \\ 1 & {x}_{2} & {x}_{2}^{2} & … & {x}_{2}^{n} \\ ⋮ & ⋮ & ⋮ & ⋱ & ⋮ \\ 1 & {x}_{m} & {x}_{m}^{2} & … & {x}_{m}^{n}\end{matrix} ] \cdot [ \begin{matrix}{a}_{0} \\ {a}_{1} \\ {a}_{2} \\ ⋮ \\ {a}_{m}\end{matrix} ] \qquad ( 4 )$$

$$X = [ \begin{matrix}1 & {x}_{0} & {x}_{0}^{2} & … & {x}_{0}^{n} \\ 1 & {x}_{1} & {x}_{1}^{2} & … & {x}_{1}^{n} \\ 1 & {x}_{2} & {x}_{2}^{2} & … & {x}_{2}^{n} \\ ⋮ & ⋮ & ⋮ & ⋱ & ⋮ \\ 1 & {x}_{m} & {x}_{m}^{2} & … & {x}_{m}^{n}\end{matrix} ] , Y = [ \begin{matrix}{y}_{0} \\ {y}_{1} \\ {y}_{2} \\ ⋮ \\ {y}_{m}\end{matrix} ] , and A = [ \begin{matrix}{a}_{0} \\ {a}_{1} \\ {a}_{2} \\ ⋮ \\ {a}_{m}\end{matrix} ]$$

are set to obtain formula Y=XA.

Next, by using a least square method, a formula for a sum of squared errors is set as follows:

$$E = {( Y - XA )}^{T} \cdot ( Y - XA ) ;$$

the formula for a sum of squared errors is simplified as follows:

$$E = {Y}^{T} Y - {Y}^{T} XA - {A}^{T} {X}^{T} Y + {A}^{T} {X}^{T} XA ;$$

and
then,

$$\frac{\partial E}{\partial A} = 0$$

is set, whereby an error between obtained data and actual data is the least, which is represented by formula (5) as follows:

$$0 - {X}^{T} Y - {X}^{T} Y + {X}^{T} XA + {X}^{T} XA = 0 \qquad ( 5 )$$

$${X}^{T} XA = {X}^{T} Y$$

$$A = {( {X}^{T} X )}^{- 1} {X}^{T} Y$$

So far, a curve equation Y=XA of the lens 10 relative to the rod portion 20 can be obtained by using the least square method, that is, a coefficient aⱼ may be defined.

It may be learned from formula (1) that the y-axis coordinate of the lens 10 may be calculated through the x-axis coordinate of the lens 10, and it may be learned from formula (2) that the x-axis coordinate of the lens 10 may be calculated through the rotation angle θ of the rotating wheel 40. A simplified formula of a value of the x-axis coordinate of the lens 10 and θ is set as X=ΘB, where

$$X , [ \begin{matrix}{x}_{0} \\ {x}_{1} \\ {x}_{2} \\ ⋮ \\ {x}_{m}\end{matrix} ] , Θ = [ \begin{matrix}1 & {θ}_{0} & {θ}_{0}^{2} & … & {θ}_{0}^{n} \\ 1 & {θ}_{1} & {θ}_{1}^{2} & … & {θ}_{1}^{n} \\ 1 & {θ}_{2} & {θ}_{2}^{2} & … & {θ}_{2}^{n} \\ ⋮ & ⋮ & ⋮ & ⋱ & ⋮ \\ 1 & {θ}_{m} & {θ}_{m}^{2} & … & {θ}_{m}^{n}\end{matrix} ] , and B = [ \begin{matrix}{b}_{0} \\ {b}_{1} \\ {b}_{2} \\ ⋮ \\ {b}_{m}\end{matrix} ] .$$

Formula (6) is obtained by using the least square method, which is as follows:

$$B = {( {Θ}^{T} Θ )}^{- 1} {Θ}^{T} X \qquad ( 6 )$$

A coefficient bⱼ may be defined, a parameter of a function x=g(θ) with respect to the rotation angle θ may be obtained, and a formula may be obtained by combining formula (1) and formula (2):

y=f(g(θ)).

This polynomial function is continuous and differentiable, so:

$$k = \frac{\partial f ( x )}{\partial x} = {f}^{′} ( x ) .$$

When the rotation angle of the rotating wheel 40 is θᵢ, the coordinates of the lens 10 relative to the rod portion 20 are (g(θᵢ), f(g(θᵢ)),0), a normal direction of this point is a direction angle of the lens 10, and a normal equation of a direction of the lens 10 is represented by formula (7) as follows:

$$y = \frac{{x}_{i} - x}{{k}_{i}} + {y}_{i} \qquad ( 7 )$$

where xᵢ=g(θᵢ), kᵢ=f′(g(θᵢ)), and yᵢ=f(g(θᵢ)).

In conclusion, when the rotation angle of the rotating wheel 40 is θ>0, the pose of the lens 10 relative to the rod portion 20 may be obtained according to the value of the rotation angle θ. The lens 10 can only move in a plane where a certain radial direction of the rod portion 20 is located, and a movable path of the lens 10 is symmetrical with respect to a central axis of the rod portion 20, so the pose of the lens 10 is symmetrical with respect to the central axis of the rod portion 20 when the rotation angle of the rotating wheel 40 θ<0 and the rotation angle θ>0.

In some embodiments, the orthopedic endoscope system controls the pull wires 41 to adjust a bending angle of the bendable portion 21 through the rotating wheel 40, the angle detection component 51 detects and sends the rotation angle of the rotating wheel 40 to the processor 90, and the processor 90 calculates the pose of the lens 10 relative to the rod portion 20 through information of the rotation angle, whereby the orthopedic endoscope system is configured to search for lesions more accurately, and medical safety risks are reduced.

The orthopedic endoscope system according to this embodiment further includes an optical navigation component 72 and an electromagnetic navigation component 81. Both the optical navigation component 72 and the electromagnetic navigation component 81 are electrically connected to the processor 90. The optical navigation component includes a reference frame 70 and a reflection sphere 71 arranged on the handle 30, and the electromagnetic navigation component 81 includes an electromagnetic positioning sensor 80 arranged at the front end of the bendable portion. The processor 90 adjusts an origin of the optical navigation component based on pose information of the lens 10, whereby a field of view of the optical navigation component is consistent with a field of view of the lens 10, and navigation accuracy is ensured. The electromagnetic navigation component 81 provides reference coordinates for the lens 10. When a difference value between the reference coordinates and the coordinates of the lens 10 is too large, a doctor is reminded of detecting a damage to the orthopedic endoscope system to ensure surgical safety.

In some embodiments, the optical navigation component 72 includes an IR Camera (Infrared Ray Camera) 73, which is electrically connected to the processor 90. The IR camera 73 is provided with an infrared emitter. The infrared ray emitted by the infrared emitter could be reflected off the reflection sphere 71 and bones. The reflected infrared ray will be received by an infrared receiver of the IR Camera 73, generating some electrical signal. The processor 90 determines the position of the reflection sphere 71 and bones, and makes an optical coordinate. In some embodiments, the infrared receiver includes infrared sensor. In some embodiments, the optical navigation component 72 includes three or more reflection spheres.

In some embodiments, the processor 90 may build a first coordinate system based on the images captured by the lens 10, and it builds a second coordinate system (i.e., an optical coordinate system) based on the optical navigation component 72. Since the optical navigation component 72 is fixed to the handle, the positions of the reflection spheres 71 are fixed relative to it. Therefore, the processor 90 determines the positions of the reflection spheres 71 using the IR Camera 73 and establishes the second coordinate system based on these positions. Furthermore, the original position and orientation of the lens 10 are recorded in the processor 90. Consequently, the processor acquires the coordinates of the lens 10 within the second coordinate system.

As the lens 10 moves, the processor receives rotation angle information obtained by the angle detection component, calculates the pose of the lens 10 relative to the rod portion 20 based on the rotation angle information, and determines the pose of the lens in the second coordinate system. In some embodiments, the position of the lens 10 differs from the position of the IR Camera, hence, the origin of the first coordinate system, which is based on the position of the lens 10, differs from the origin of the second coordinate system, which is based on the position of the IR Camera. Therefore, the processor 90 adjusts the origin of the second coordinate system based on the pose information of the lens and the optical navigation component 72 to align it with the origin of the first coordinate system. For example, the processor can set the position of the lens 10 as the origin of the second coordinate system so that the field of view of the optical navigation component is consistent with the field of view of the lens 10.

In some embodiments, the processor 90 calculates the position of the bones that appear in images captured by the lens 10 within the first coordinate system. Furthermore, the processor 90 can also calculate the positions of the bones and the reflection spheres 71 within the second coordinate system, based on the optical navigation component 72. Therefore, the processor 90 can perform a coordinate fitting of the first and second coordinate systems to calculate more accurate bone positions.

In some embodiments, the position of the electromagnetic positioning sensor 80 is determined by an external magnetic field to the human body. When this magnetic field passes through the electromagnetic positioning sensor 80, a weak electrical signal is generated by the coil within the sensor, which is then transmitted to the processor 90 via a wire. The processor 90 acquires the position of the electromagnetic positioning sensor 80 by calculating this electrical signal. The position of the electromagnetic positioning sensor 80 relative to the lens is fixed. Therefore, the processor 90 can calculate the position of the lens, and thus the electromagnetic navigation component provides reference coordinates for the lens.

It is to be noted that a method for fitting positioning information of an optical navigation technology and/or an electromagnetic navigation technology and/or an endoscope system navigation technology is a conventional technology. Therefore, a specific principle of navigation is not described in detail in this embodiment. It is to be noted that the lens 10 of this embodiment is represented by using a right-handed coordinate system.

Various technical features of the above embodiments may be arbitrarily combined. To make the description concise, all possible combinations of the various technical features in the foregoing embodiments are not described. However, contradiction in the combinations of these technical features are considered to be in a range described in this specification as long as there is no conflict.

The above embodiments merely express several implementations manners of the present disclosure, with specific and detailed description, and are not to be construed as a limitation on the patent scope of the present disclosure. It is to be noted that a number of variations and modifications may be made by those of ordinary skill in the art without departing from the conception of the present disclosure. All of these belong to the scope of protection of the present disclosure. Therefore, the scope of protection of the present disclosure should be determined by the appended claims.

What is claimed is:

1. A steerable orthopedic endoscope system with navigation function, comprising:
a handle;
an endoscope body, comprising a rod portion connected to the handle, a bendable portion located at a front end of the rod portion, and a lens located at a front end of the bendable portion, wherein the bendable portion is internally provided with bending sections, whereby the bendable portion is capable of bending at different angles;
a rotating adjustment component, comprising a rotating wheel and two pull wires arranged on two sides in a radial direction of the rotating wheel respectively, wherein the two pull wires are connected to the bending sections to adjust a bending degree of the bendable portion;
an angle detection component, comprising a marker arranged on the rotating wheel and a detection device configured to identify the marker, wherein the detection device obtains a rotation angle of the rotating wheel by detecting changes of the marker; and
a processor, electrically connected to the angle detection component and the rotating adjustment component, wherein the processor receives rotation angle information obtained by the angle detection component, and calculates a pose of the lens relative to the rod portion according to the rotation angle information, and the pose comprises coordinates and a direction angle of the lens.

2. The steerable orthopedic endoscope system with navigation function according to claim 1, wherein an operation in which the processor calculates the pose of the lens relative to the rod portion according to the rotation angle information comprises:
determining an x-axis coordinate (x) and a y-axis coordinate (y) of the lens according to the following mathematical expression:
$$y = {\sum }_{j = 0}^{n} {a}_{j} \cdot {x}^{j} = f ( x )$$
$$x = {\sum }_{j = 0}^{n} {b}_{j} \cdot {θ}^{j} = g ( θ )$$
wherein n is a number of terms in a polynomial, aⱼ and bⱼ are respectively coefficients of a j^{th} item in a vertical coordinate and a horizontal coordinate, and θ is the rotation angle of the rotating wheel.

3. The steerable orthopedic endoscope system with navigation function according to claim 2, wherein the operation in which the processor calculates the pose of the lens relative to the rod portion according to the rotation angle information further comprises:
determining a normal at the lens according to the following mathematical expression, and taking an angle of the normal as the direction angle of the lens:
$$y = \frac{{x}_{i} - x}{{k}_{i}} + {y}_{i}$$
wherein xᵢ=g(θᵢ), kᵢ=f′(g(θᵢ)), and yᵢ=f(g(θᵢ)).

4. The steerable orthopedic endoscope system with navigation function according to claim 1, comprising an optical navigation component, wherein the processor adjusts an origin of the optical navigation component based on pose information of the lens, whereby a field of view of the optical navigation component is the same as a field of view of the lens.

5. The steerable orthopedic endoscope system with navigation function according to claim 4, wherein the optical navigation component comprises a reference frame and a reflection sphere arranged on the handle.

6. The steerable orthopedic endoscope system with navigation function according to claim 5, wherein the optical navigation component comprises a plurality of the reflection spheres, and a number of the reflection spheres is three or more.

7. The steerable orthopedic endoscope system with navigation function according to claim 1, wherein the marker is a grating, and the detection device is a sensor that matches the grating.

8. The steerable orthopedic endoscope system with navigation function according to claim 1, wherein the rotating wheel is driven by a motor, and the motor is embedded into the handle.

9. The steerable orthopedic endoscope system with navigation function according to claim 8, wherein a button configured to start or stop the motor is arranged on the handle.

10. The steerable orthopedic endoscope system with navigation function according to claim 1, comprising an electromagnetic navigation component, providing reference coordinates for the lens, and the electromagnetic navigation component comprises an electromagnetic positioning sensor arranged at the front end of the bendable portion.

11. The steerable orthopedic endoscope system with navigation function according to claim 2, wherein n, aⱼ and bⱼ are determined by fitting a plurality of calibration results.
